(19) United States
(12) Patent Application Publication
(10) Pub. No.: US 2012/0009242 A1
(43) Pub. Date: Jan. 12, 2012

(54) BLOOD COAGULATION INDUCING POLYMER HYDROGEL

(75) Inventors: Brendan J. Casey, College Park, MD (US); Peter Kofinas, North Bethesda, MD (US); Adam Behrens, Olney, MD (US); Trevor A. Snyder, Edmond, OK (US); Bartley P. Griffith, Gibson Island, MD (US)

(73) Assignees: UNIVERSITY OF MARYLAND, COLLEGE PARK, College Park, MD (US); UNIVERSITY OF MARYLAND, BALTIMORE, Baltimore, MD (US)

(21) Appl. No.: 13/127,152

(22) PCT Filed: Nov. 3, 2009
(86) PCT No.: PCT/US2009/063049

§ 371 Date: Sep. 19, 2011

Related U.S. Application Data
(60) Provisional application No. 61/110,698, filed on Nov. 3, 2008.
(60) Provisional application No. 61/234,773, filed on Aug. 18, 2009.

(51) Int. Cl. A61K 31/785; A61L 15/24; A61P 7/04; A61L 15/44; A61L 31/12; A61L 31/16; C08F 226/02; A61L 15/58

(52) U.S. Cl. 424/446; 526/307; 424/78.35; 424/447; 424/445; 424/400

(57) ABSTRACT

The present application is drawn to a synthetic, polymer hydrogel-based material, which is able to actively induce the body's natural hemostatic coagulation process in blood or acellular plasma. The present invention provides the development of a primary amine containing polymer hydrogel capable of inducing blood coagulation and delivering therapeutics for hemostatic or wound care applications, and a method of forming such a primary amine containing polymer hydrogel capable of inducing the blood coagulation process. The primary amine containing polymer hydrogel is able to achieve the same end result as biological-based hemostatics, without the innate risk of disease transmission or immunological response, and at a fraction of the price. Furthermore, due to its inherent hydrogel-based design the material has the capability of arresting blood loss while simultaneously delivering therapeutics in a controlled manner, potentially revolutionizing the way in which wounds are treated.

### CROSS-REFERENCE TO RELATED APPLICATIONS

This application claims priority to U.S. provisional application Ser. Nos. 61/110,698, filed Nov. 3, 2008 and 61/234,773, filed Aug. 18, 2009, which are incorporated in their entireties by reference herein.

### STATEMENT REGARDING FEDERALLY SPONSORED RESEARCH OR DEVELOPMENT

This invention was made with government support under CTS0640778 awarded by the National Science Foundation. The U.S. Government has certain rights in this invention.

### TECHNICAL FIELD

The present invention relates to a polymer material capable of actively inducing the blood coagulation cascade while simultaneously delivering therapeutics in a controlled manner for application in the wound care field.

### BACKGROUND

Technology capable of effectively controlling traumatic hemorrhaging does not exist. As a result, millions of people around the world are dying every year. Over 3,000,000 worldwide, 156,000 in the U.S. alone, die from trauma related injuries each year. Half will die before they reach the hospital, and 80% will die within 24 hours of hospital admission. Uncontrolled hemorrhaging, or blood loss, accounts for almost half of these deaths. Moreover, on the battlefield an astounding 85% of military mortalities are due to blood loss, a statistic that has remained mostly unchanged since the Vietnam War. Army medics, emergency medical technicians (E.M.T.) personnel, and other various emergency responders need a product capable of providing rapid and effective hemostasis until they can get their patients to the operating room. Once at the operating room, hemostatic products are still crucial to surgeons who must control bleeding to maintain the stability of their patient.

Current products are either exorbitantly expensive, ineffective, and/or have adverse side effects. There is a clear need for a hemostatic product which is inexpensive, effective, with little to no adverse side effects and medics, emergency responders, and surgeons are all looking for a solution. Furthermore, there is currently no existing product or material on the market with the capability of providing rapid hemostasis along with simultaneous drug delivery. A product which could effectively stop blood loss while simultaneously delivering potentially life-saving therapeutics such as adrenaline or insulin in a regulated manner would have a multitude of applications from the battlefield to the operating room.

Death may occur in minutes after a traumatic injury due to blood loss. The body has natural mechanisms to control hemorrhaging yet these processes may be insufficient in cases of excessive hemorrhaging, defective due to medical conditions such as hemophilia, or compromised due to adverse effects of medication like Coumadin. The natural hemostatic response is not adequate to control major hemorrhaging due to traumatic injury, which is the main reason why such injuries, if gone untreated, are typically fatal. Administration of biologically derived blood products to augment the native hemostatic response and to maintain adequate oxygen delivery to the brain and vital organs, carries significant risks including disease transmission, infection, pulmonary dysfunction, and immune response. Furthermore, many people have deficiencies within their hemostatic response i.e. hemophilia, which prevent them from adequately stopping blood loss. Millions of people around the world suffer from bleeding disorders, and are unable to clot blood effectively. Current treatments are typically limited to clotting factor (Factor VIII, Factor II) replacement therapies, which are typically painful and exorbitantly expensive. There is a clear need for an inexpensive, painless alternative to current treatments. Whether the injury overwhelms the body's clotting response or the native response is deficient or compromised, an inexpensive, synthetic material which has the ability to induce clotting effectively, while simultaneously delivering therapeutics would undoubtedly revolutionize the way in which wounds are treated.

The field of hemostatic agents and materials has expanded dramatically within the last decade. This considerable expansion and evolution of the field, throughout the last decade has also been accompanied by tremendous diversification resulting in a multitude of hemostatic products now available on the market, each with their own advantages and disadvantages. The hemostatic products currently available on the market today are either biological-based or synthetic-based. Biological-based hemostatics are comprised of animal or “animal derived” substrates which are able to initiate, amplify, and/or assist the natural coagulation response. Although they have excellent hemostatic effects and work via the promotion of the body's natural responses, they are incredibly expensive (up to $500 per application) and carry risks of disease infection and severe immunological response. Synthetic hemostatic agents are typically less expensive and immune inert yet often fail to effectively induce the coagulation cascade. Such synthetic agents are mainly designed to be mere physical obstructions to impede blood flow while providing a scaffold for the coagulation process to occur. A purely synthetic, polymer-based hydrogel material capable of effectively inducing the body's natural coagulation response has enormous potential within the field. Such a material is unique to the market in that it could be used to effectively stop bleeding while simultaneously delivering necessary therapeutics to a wound site.

### SUMMARY OF THE INVENTION

The inventors of the present application have developed a purely synthetic, polymer hydrogel-based material, which is able to actively induce the body's natural hemostatic coagulation process in blood or acellular plasma. There is currently no polymer hydrogel-based, synthetic hemostatic agent with the capability of inducing the formation of a natural hemostatic matrix in the absence of platelets or blood cells. Since the material is able to induce the formation of a natural hemostatic plug in the absence of platelets or cells, it has enormous potential as a hemostatic agent in surgery, to treat trauma victims, and especially for patients with platelet disorders. The material is able to achieve the same end result as biological-based hemostatics, without the innate risk of disease transmission or immunological response, and at a fraction of the price. Furthermore, due to its inherent hydrogel-based design the material has the capability of arresting blood loss while simultaneously delivering therapeutics in a controlled manner, potentially revolutionizing the way in which wounds are treated.

The blood coagulation cascade may be activated via two distinct routes, the tissue factor pathway and the intrinsic pathway, also known as the contact activation pathway. Both pathways eventually result in the activation of a common pathway, which leads to the formation of a fibrin-based hemostatic clot Our research has shown that a material is able to induce the formation of fibrin via the tissue pathway factor Specifically, a positively charged polymer network with adequate mechanical rigidity is capable of efficiently and effectively inducing the activation of FVII, which in turn leads to the activation of the common pathway and subsequent fibrin formation. Furthermore, the material is able to induce the activation of FVII irrespective of calcium or platelets which are typically vital cofactors of the process.

In one aspect, the present invention provides a cross-linked primary amine containing polymer hydrogel capable of inducing blood coagulation, and subsequent fibrin clot formation, while simultaneously delivering therapeutics in a controlled or regulated manner for wound care applications.

In another aspect the present invention is able to induce coagulation in Factor XII, XI, IX, VIII, and V deficient blood plasma.

In another aspect the present invention is capable of inducing the activation of FVII irrespective of calcium and platelets.

In another aspect, the present invention provides a method of forming the specific type of cross-linked primary amine containing polymer hydrogel in order to effectively induce blood coagulation: (a) adding either a primary amine containing monomer or primary amine containing polymer with a pKa greater than 7.4 thereby being positively charged within a plasma or blood environment (pH 7.4); (b) addition of additional monomers different from the initial primary amine containing monomer; (c) forming a polymer matrix by initiating polymerization of monomer units into polymer strand; (d) cross-linking the polymer strands to produce a polymeric mesh network.

In other aspects, the present invention provides various uses for a polymer hydrogel capable of inducing blood coagulation and delivering therapeutics in a controlled manner, in the health care field.

### BRIEF DESCRIPTION OF THE DRAWINGS

FIG. 1: Coagulate complex formed by optimal hydrogel after immersion in citrated plasma. Typical coagulate complex (fibrin-hydrogel complex) formed after rotating the optimal hydrogel in human plasma (4% w/v sodium citrate) for 18 hours.

FIG. 2: Characterization images (H&E, IHC, ESEM) of coagulate complex formed by optimal hydrogel after immersion in citrated plasma. (A) H&E stained micrograph image of coagulate complex. Polymer hydrogel appears as lighter, smoother material on right side of the micrograph while fibrin appears as the darker, rougher material on the left side of them micrograph. (B) IHC stained micrograph image of coagulate complex. (C) ESEM surface image of the coagulate complex.

FIG. 3: Optimization experiment. Experiment aimed to investigate the dependence of fibrin formation on various compositional factors including total monomer concentration (acrylamide+APM+BIS), positive electrostatic charge (APM), and cross-linker ratio (acrylamide:APM:BIS). Acrylamide concentration is located on the horizontal axis while APM concentration is on the vertical axis. BIS concentration is also indicated on the horizontal axis and is kept constant for each respective acrylamide concentration. The amount of fibrin formation induced by each composition was visually scored from 0 (no fibrin formation) to 10 (substantial fibrin formation). All samples were run in triplicate.

FIG. 4. Factor deficient and factor inhibited plasma experiment. Optimal hydrogel composition tested in various factor deficient and factor inhibited plasmas. The resulting fibrin formation was visually scored from 0 (no fibrin formation) to 10 (substantial fibrin formation) and graphed accordingly. All samples were run in triplicate.

FIG. 5: Kinetic biological mechanism experiments. Factor VIIa concentration (A), calcium concentration (B), and TFPI activity (C) was measured in human plasma containing various hydrogel compositions at 30, 90, and 180 minutes (left axis: bar graph). The amount of fibrin formation was also rated for each composition at each time point (right axis: line graph). Data is representative of an average and corresponding standard deviation (error bar) of three (n=3) separate sample trials. Asterisk (*) indicates duplicate sample point.

FIG. 6: Dynamic mechanical analysis. Dynamic mechanical analysis of three compositions used in the kinetic biological mechanism experiments (FVIIa, calcium, TFPI) ranging from high APM, low acrylamide and BIS content (composition A) to low APM, high acrylamide and BIS content (composition F). Spectra for sample compositions C and F are shifted vertically to avoid overlapping of data.

FIG. 7: Fresh sheep blood experiment. 250 mg of our hydrogel material (A) compared to a control (B). Clotting time of blood with material was dramatically decreased (˜45 seconds) compared to control (˜10 minutes).

FIG. 8: Prototype images. (A) Computer generated graphic of prototype used in animal experiment. (B) Actual prototype used in animal trial.

FIG. 9: Animal trial. (A) Image of lung at time of incision, before hydrogel was applied. (B) Image of the incision site after the hydrogel prototype bandage was applied for approximately 2 minutes.

FIG. 10: Stained lung section of incision site. Micrograph of hematoxylin and eosin (H&E) stained section of incision site after hydrogel material was applied for two minutes.

### DETAILED DESCRIPTION OF THE INVENTION

A reference to an element by the indefinite article “a” or “an” does not exclude the possibility that more than one of the element is present. Rather, the article “a” or “an” is intended to mean one or more (or at least one) unless the text expressly indicates otherwise. The terms “first,” “second,” and so on, when referring to an element, are not intended to suggest a location or ordering of the elements. Rather, the terms are used as labels to facilitate discussion and distinguish elements from one another.

The foregoing description and examples have been set forth merely to illustrate the invention and are not intended to be limiting. Each of the disclosed aspects and embodiments of the present invention may be considered individually or in combination with other aspects, embodiments, and variations of the invention. Modifications of the disclosed embodiments incorporating the spirit and substance of the invention may occur to persons skilled in the art and such modifications are within the scope of the present invention.

The present invention provides the development of a primary amine containing polymer hydrogel capable of inducing blood coagulation and delivering therapeutics for hemostatic or wound care applications. Various therapeutics intended to be delivered include but are not limited to ester or amide based anesthetics such as Novocain or Lidocain, antibiotics such as erythromycin or bacitracin, vasoconstrictors such as adrenaline, and pain relievers or anti-inflammatory medicines such as topricin, acetaminophen, or ibuprofen. The hydrogel may be designed in order to deliver the drugs in various ways including based on a swelling change, a change in pH, or via the introduction of a magnetic or electric field.

The present invention provides the development of a primary amine containing polymer hydrogel capable of inducing blood coagulation and delivering other hemostatic agents for hemostatic or wound care applications, including biological-based hemostatic agents and non biological-based hemostatic agents.

Biological-based hemostatics contain, incorporate, or are derived from biological substrates, i.e. proteins, or cells. They can further be subdivided into the type of biological substrates incorporated into the system including collagen, thrombin, fibrin, albumin, and/or platelets.

Collagen is the main protein of connective tissue in mammals, including the skin, bones, ligaments, and tendons making up about 30% of the total protein in the body. In addition to providing structural integrity for the animal body, including all organs, collagen also activates the contact activation pathway of the coagulation cascade. Due to collagen's ability to induce coagulation, along with the fact that is it naturally occurring, makes it an ideal choice for a hemostatic agent. Collagen is typically incorporated into the products via gelatin or microfibrillar form. Gelatin is an irreversibly hydrolyzed form of collagen and may be prepared as a powder, sponge, sheet, film or foam. Gelatin products are typically pliable, easy to handle, and relatively inert. When placed in soft tissue gelatin products typically absorb in 4 to 6 weeks, yet when applied to bleeding nasal, rectal or vaginal mucosa, will liquefy in approximately 2 to 5 days. The product Gelfoam (Pfizer, New York, N.Y.), first introduced in 1945, is produced from purified pork skin gelatin granules. The foam swells up to 45 times its original dry weight and 200% of its initial volume.

Microfibrillar collagen is the predominant form used in modern hemostatic products. The collagen network acts as a framework which aggregates clotting factors, platelets, along with various coagulative and adhesive proteins to facilitate clot formation. Furthermore, the collagen fibrils are able to efficiently activate the contact activation coagulation pathway. The product is typically formed into various products including powder (shredded fibrils), sheets, and sponges. Market examples include Ultrafoam and Avitene (Davol Inc., Cranston, R.I.), Instat (Johnson & Johnson, Langhorne, Pa.), Helistat and Helitene (Integra LifeSciences, Plainsboro, N.J.), Collatape/CollaCote/CollaPlug (Integra Lifesciences Corporation, Plainsboro, N.J.), Collastat and Collatene (Xemax, Napa, Calif.).

Collagen-based hemostatic products are easily removable, cause little aggravation to the wound site, and can be very effective hemostats (especially relative to cellulose or gelatin hemostats). Disadvantages of collagen products include their prohibitive high price (around $150 per dressing), poor biodegradability, inherent risk of antigenicity, low solubility (difficult to make concentrated solutions), and handling difficulties since the products will irreversibly adhere to any hydrated surface.

Thrombin is the central activating enzyme of the common coagulation pathway. Thrombin circulates within the blood in its precursor, or zymogen form, prothrombin. Prothrombin is specifically cleaved to produce the enzyme thrombin. The main role of thrombin in the coagulation pathway is to convert fibrinogen into fibrin, which in turn is covalently cross-linked to produce a hemostatic plug. Thrombin-based products are typically sold in liquid or powder form and include Thrombostat (ParkeDavis, Ann Arbor, Mich.), Thrombin-JMI (King Pharmaceuticals, Briston, Tenn.), and Quixil (Omrix Biopharmaceuticals Ltd, Tel Hashomer, Israel). There are also several combination products which include Evicel (Johnson & Johnson, Langhorne, Pa.) which is a combination of thrombin and fibrin used mainly as a tissue sealant, along with FloSeal (Baxter Healthcare Corporation, Westlake Village, Calif.) and SurgiFlow which are both hybrid products composed of bovine or porcine gelatin and thrombin.

Thrombin-based products take advantage of the natural physiologic coagulation response by augmenting, amplifying, and assisting the process. Advantages of these products include low risk of foreign body or inflammatory reactions, firm attachment to wound bed, and its excellent hemostatic effect, specifically with patients that have platelet dysfunctions. Another advantage of thrombin is the versatility that the product may be applied, in powder or liquid (spray on) form. Disadvantages of these products include their often prohibitive high price ($75-$300 per application), difficulty of use including the inconvenience of premixing preparation, along with the risk of intravenous introduction which may result in intravascular clotting.

Fibrin is a fibrillar protein which is polymerized and cross-linked to form a mesh network, typically at the site of an injury after the induction of the coagulation cascade. The mesh network, incorporative of other various proteins and platelets, forms a hemostatic plug to prevent continuous or further blood loss. Fibrin is activated from its inert zymogen, fibrinogen, by thrombin. Fibrin is in turn polymerized and covalently cross-linked by another coagulation factor, known as Factor XIIIa. Due to its natural mechanical hemostatic role fibrin has been commercially used to control blood flow since the early 1900s. Most fibrin glues or fibrin sealants are derived from human and bovine proteins. The product is typically sold in the form of a dual syringe. The first syringe compartment contains the matrix and matrix stabilizing components including fibrinogen, factor XIII, fibronectin, and fibrinolysis inhibitors. The second syringe compartment contains the activating agent, typically thrombin and calcium chloride. At the time of application, the contents of both syringes are ejected, combining to activate fibrin matrix formation which typically takes a matter of seconds to set and approximately 5 to 10 days to degrade or absorb into the body. Various fibrin sealants on the market include Tiseel (Baxter HealthCare Corporation, Westlake Village, Calif.), FibRx (CryoLife Inc., Kennesaw, Ga.), Crosseel (Johnson & Johnson, Langhorne, Pa.), Hemaseel (Haemacure Corporation, Montreal, Quebec), Beriplast P (Aventis Behring, King of Prussia, Pa.), and Bolheal (Kaketsuken, Kumamoto, Japan).

Fibrin-based hemostatics or tissue sealants are fast-acting, composed of native coagulative factors, are biodegradable, do not promote inflammation or tissue necrosis, have diverse applications, and are particularly useful in patients with coagulation deficiencies such as hemophilia or von Willebrand's disease. Major disadvantages of fibrin-based hemostatics include their often prohibitive price ($100-$300/mL), their fragile nature, and difficulty of handling and application.

Other prominent biological-based hemostatic products include those composed of covalently cross-linked protein networks such as BioGlue (Cryolife, Kennewsaw, Ga.), along with products which incorporate platelets such as Costasis, marketed as Vitagel (Orthovita, Malvern, Pa.). BioGlue is comprised of bovine serum albumin, and various other proteins, cross-linked with glutaraldehyde to form a rigid, insoluble matrix. The reaction occurs spontaneously upon the introduction of glutaraldehyde to the protein mixture, and requires no external factors such as coagulation factors. Disadvantages of the product include the high price ($300-$425/5 ml application), mediocre hemostatic effect, necessity of a dry environment for application, the toxic effects associated with tissue exposure to glutaraldehyde, and risk of immune reactions associated with glutaraldehyde-based products.

Costasis is a combination product combining bovine collagen and the patient's own platelets. The collagen within the product promotes the initiation of the contact activation pathway of the coagulation cascade. The presence of platelets in such a product improves overall clot strength and supplies various growth factors which facilitate tissue regeneration. Disadvantages include high price ($100-$150/mL) and difficulty of application.

Non biological-based, or synthetic, hemostatic agents are defined as any products which do not incorporate biological materials, or more specifically animal derived components. Synthetic hemostatics are typically cheaper, easier to use, and easier to apply relative to their biological counterparts. Furthermore, synthetic hemostatics have no innate antigenicity, rarely induce immune responses or inflammatory reactions, and are inherently free of disease vectors.” The main classes of synthetic hemostatics include cyanoacrylates, polysaccharides (e.g. oxidized cellulose, N-acetyl glucosamine), synthetic polymers, and mineral/metal based.

Cyanoacrylates are liquids that rapidly polymerize. These products create a tight seal between tissues, obstructing blood flow. Cyanoacrylates are categorized upon their length. Shorter chain cyanoacrylates (ethyl cyanoacrylates) are typically quicker to absorb yet more toxic relative to intermediate (butyl cyanoacrylates) or longer chain cyanoacrylates (octyl cyanoacrylates). Due to their inherent high toxicity few hemostatic products composed of short chain cyanoacrylates have reached the market. There is however some research supporting the efficacy of Krazy Glue (ethyl-2-cyanoacrylate, Elmer's, Columbus, Ohio) for cutaneous wound closure. Cohera Medical Corporation is currently in the process of developing a butyl cyanoacrylate (isobutyl-2-cyanoacrylate), marketed as TissuGlu (Cohera Medical Inc., Pittsburgh, Pa.). Furthermore, there are currently several octyl acrylate-based hemostatic products that are FDA-approved for skin closure which include Dermabond (Ethicon, Somerville, N.J.) and Band-Aid Liquid Bandage (Johnson & Johnson, Langhorne, Pa.).

Cyanoacrylates are typically nonreactive, do not promote infection, are rapidly curing, and are only moderately expensive. Disadvantages of cyanoacrylates and cyanoacrylate-based hemostatics include difficulty of application due to their highly adhesive nature, and risk of tissue neurotoxicity, fibrosis and inflammatory reactions.

The two main polysaccharides used as hemostatics today are oxidized cellulose and poly-N-acetyl glucosamine. The hemostatic effects of certain polysaccharides, specifically oxidized cellulose and N-acetyl glucosamine, have been known since the early twentieth century. Oxidized cellulose is derived from plant fiber, which is in turn oxidized in the presence of nitrogen dioxide to form cellulosic acid. Oxidized cellulose activates the coagulation cascade (contact activation pathway) and accelerates thrombin generation within the body. Furthermore, the polysaccharide meshwork serves as a physical framework for coagulation to occur, with moderate absorbent properties. Oxidized cellulose products on the market today include Oxycel (Becton Dickinson, Franklin Lakes, N.J.), Celox (Medtrade Products Ltd., Crewe, England), Surgicel (Ethicon Incorporation), and BloodStop (LifeScience PLUS, Inc., Santa Clara, Calif.).

Cellulose-based hemostatics are relatively easy to handle, fully absorbable and biodegradable (over 1 to 6 weeks), relatively inexpensive, and have antibacterial properties. The major drawback of these products is the risk of foreign body reactions. Furthermore, these products have only moderate coagulation-inducing capability and therefore are reserved as an adjunct to the natural response rather than a synthetic replacement.

Poly-N-acetyl glucosamine, also known as chitin or chitosan, is a complex polysaccharide produced by fermenting microalgal cultures. The hemostatic effects of poly-N-acetyl glucosamine are believed to be a result of the attraction and binding of circulating blood cells. The positive charges on the polymer attract the negatively charged erythrocytes, to help seal the clot. Poly-N-acetyl glucosamine also has vasospasm effects. Poly-N-acetyl glucosamine products include HemCon (HemCon Inc., Portland, Oreg.), TraumaDex (Medafor, Minneapolis, Minn.), SyvekPatch (Marine Polymer Technologies Inc., Danvers, Mass.), Clo-Sur P.A.D. (Scion Cardio-Vascular, Miami, Fla.), and Chito-Seal (Abbott Vascular Devices, Redwood, City, Calif.).1

Advantages of poly-N-acetyl glucosamine dressings include their ease of application, robustness, and lack of toxicity. Disadvantages include the high cost ($100 per unit), and variability of efficacy between batches.

Most polymer-based hemostatics are designed to provide a mechanical tissue sealant. The majority of products on the market today are composed of polyethylene glycol (PEG) which are applied and polymerized at the wound site. The polymer is typically cross-linked with itself or with a primer to yield a robust framework stopping blow flow and sealing tissue. Most PEG products undergo biodegradation in approximately 30 days. PEG products include Coseal (Baxter Healthcare Corporation, Westlake Village, Calif.) and AdvaSeal-S (Genzyme Corporation, Cambridge, Mass.). PEG-based hemostatics or tissue sealants are typically non inflammatory, do not induce immune response, and are biodegradable. Drawbacks include difficulty of application and high price ($400/application).

Pro QR Powder (Biolife, Sarasota, Fla.) is another polymer-based hemostatic on the market today. Pro QR is a combination product of a hydrophilic polymer and a potassium iron oxyacid salt. The polymer is absorptive of blood flow, promoting the formation of a natural blood clot while the potassium salt component releases iron which complexes with proteins and activates hemostatic channels. The product is inexpensive, nontoxic, easily stored, flexible, stops bleeding rapidly, and is available over the counter. The main drawback of the product is its awkward application.

The final class of non-biological, or synthetic, hemostatics includes those which incorporate metal salts or minerals such as zinc, iron, silver nitrate, or aluminum chloride. Although this class of hemostatics are typically easy to use, cost-effective, and provide adequate hemostatic effects their toxic side effects limit their appeal.

Zinc paste was first used to fix tissue after surgery in the early 1940s. Zinc paste solutions have impressive hemostatic abilities but are rarely used do to their harmful side effects including pain and toxicity of the site. Monsel's solution is a 20% ferric subsulfate solution, which is believed to occlude vessels via protein precipitation. Monsel's solution is easy to obtain, cost-effective, and resistant to bacterial contamination. Major disadvantages include its caustic and toxic nature which may promote melanocyte activity, increased erythema, dermal fibrosis, and reepithelialization. Silver nitrate is typically used as a 10% solution and coagulates blood through protein precipitation. Silver nitrate is cost-effective, easy to use, and has potent antibacterial properties. Disadvantages include its severe tissue toxicity, risk of permanent skin discoloration, and the painful burning sensation experienced upon application. Aluminum chloride has modest hemostatic properties and is prepared in concentrations of 20% to 40% in water, alcohol, ether, or glycerol. Its mechanism of action is thought to be caused by the hydrolysis of the salt, resulting in the generation of hydrogen chloride which causes vasoconstriction, and can assist in the activation of the extrinsic coagulation pathway. Aluminum chloride is cost effective, easy to use, and may be stored at room temperature. Side effects of its use include painful paresthesias, tissue irritation, and reepithelialization. Aluminum chloride solutions are marketed as Drysol and Xerac AC (person-Covey, Dallas, Tex.).I

A small subclass of hemostatics is based upon various mixtures of minerals. Zeolite is a granular mixture of silicon, aluminum, sodium, and magnesium derived from lava rock. When coming into contact with blood the mixture absorbs water, concentrating platelets and coagulation factors within the wound, accelerating the clotting process. QuikClot (Z-Medica, Wallingford, Conn.) and WoundStat (TraumaCure, Bethesda, Md.) are two main products based upon a zeolite mixture. Zeolite is inexpensive, easy to manufacture, clots fairly quickly, robust under various conditions, and is fairly immunological inert. The main drawback of the formulation is the risk of thermal injury associated with use.

A hydrogel is generically defined as an insoluble, cross-linked network of polymer chains which swells in an aqueous environment. A hydrogel may be chemically cross-linked through covalent bonds or physically cross-linked through entanglements or non-covalent interactions. Due to their unique properties hydrogels have been used in various pharmaceutical and biomedical applications. Since it is possible to create hydrogel constructs with specific degradative and swelling characteristics their potential for tissue engineering and artificial implantation is immense. Furthermore, because hydrogels can be engineered with “smart” swelling behavior based on time, pH, ionic concentration, electrical, or magnetic stimuli they have been used with incredible success as drug delivery systems.

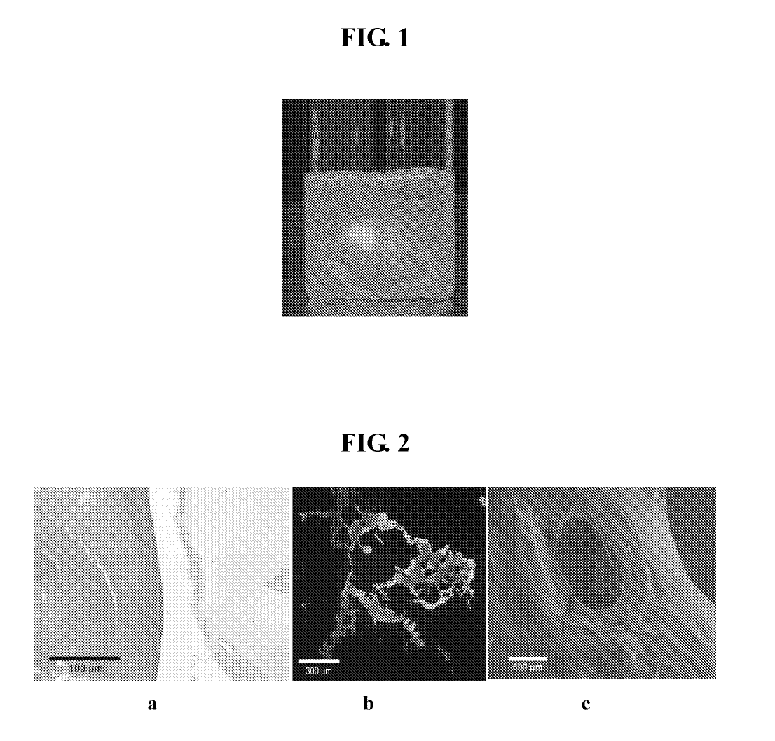

A cationic, acrylamide-based hydrogel has been developed which exhibits unique and potent coagulation-inducing effects upon the interaction with blood or acellular plasma. The hydrogel is composed of acrylamide, N-(3-Aminopropy)methacrylamide hydrochloride, and cross-linked with N-N′-methylenebisacrylamide. Upon interaction with acellular plasma the hydrogel initiates the coagulation cascade which results in the formation of a natural, fibrin-based hemostatic matrix (FIG. 1). The stained microscopic images clearly show two distinct materials; the polymer hydrogel, which appears smooth and glassy on the right side of each image and a fibrin layer, which surrounds the polymer hydrogel located on the left side of each image.

In one aspect, the present invention provides a specific method of forming such a primary amine containing polymer hydrogel capable of inducing the blood coagulation process. In one embodiment, the primary amine monomer may be a strong base (wherein its ability to exhibit a positive charge is largely pH independent). In another embodiment, the primary amine monomer may be a weak base (wherein its ability to exhibit a positive charge is largely pH dependent). In yet another embodiment, the primary amine monomer is a weak base with a pKa above 7.4 and is able to exhibit a strong positive charge at the pH of blood and plasma (˜7.4).

The method involves mixing at least one monomer with a primary amine group, along with desired other monomers, different from the initial primary amine containing monomer in a solvent, specifically an aqueous solvent. The polymer hydrogel is formed by polymerizing the monomers and cross-linking either after or during the polymerization process. Preferably, the polymer hydrogel is cross-linked in such a way so as to ensure the creation of pockets within the hydrogel which are incredibly dense with primary amine functionality. These dense pockets of positive electrostatic charge are able to induce coagulation through a Factor VII dependent mechanism. Without being bound to any specific theory, it is believed that that hydrogel acts as a catalyst activating and enhancing the functioning of Factor VII along with the Factor VH-tissue factor complex. Research has shown that this phenomenon is dependent on positive electrostatic charge and the mechanical rigidity of the hydrogel formed. That is to say, the primary amine monomer, within the hydrogel, should be positively charged at the pH of blood and/or plasma, 7.4. Therefore, if the monomer is a weak base it preferably has a pKa of at least 7.4, more preferably at least 8, and even more preferably, at least 8.5, to ensure the predominant majority of the monomers are hydrogenated bearing a positive charge. Furthermore, as stated previously the amine monomer containing polymer strand must be sufficiently cross-linked to create an appropriately rigid material.

In certain embodiments, the monomer units are capable of exhibiting an electrostatic charge in an aqueous solution. In particular the primary amine containing monomer is able to exhibit a positive electrostatic in a salt buffered, aqueous environment of pH 7.4 (blood/plasma). In some cases, the contributing monomer units may be acidic or basic, which under the appropriate pH conditions, exhibit a negative or positive electrostatic charge, respectively. The acid/base monomer units may have varying levels of acidity/basicity, which will determine the extent to which the monomer units will be present in the anionic/cationic form at the pH level of the aqueous solution. With respect to acidic monomer units, the monomer unit may be a strong acid (in which its ability to exhibit a negative charge is largely pH independent) or a weak acid (in which its ability to exhibit a negative charge is pH dependent respect to basic monomer units), the monomer unit may be a strong base (in which its ability to exhibit a positive charge is largely pH independent) or a weak base (in which its ability to exhibit a positive charge is pH dependent).

In certain embodiments, the monomer units used are able to exhibit marked morphological or structural changes based on certain stimuli such as pH, electric field, magnetic field, or temperature for regulated drug delivery applications. In some cases the contributing monomer units may be basic, which under the appropriate pH conditions, exhibit a positive electrostatic charge. The base monomer units may have varying levels of basicity, which will determine the extent to which the monomer units will be present in the cationic form at the pH level of the aqueous solution. The monomer unit may be a strong base (in which its ability to exhibit a positive charge is largely pH independent) or a weak base (in which its ability to exhibit a positive charge is pH dependent).

In some cases the contributing monomers may be electrically sensitive, that is, the monomer is able to exhibit a structural phase change upon introduction to an electrical field. Examples of such monomers include vinyl alcohol, diallyldimethylammonium chloride, and acrylic acid.

In some cases the contributing monomers may able to exhibit a marked morphological or structural change based upon temperature. An example of such a temperature sensitive monomer is N-isopropylacrylamide. The monomer may be used to produce a temperature-sensitive hydrogel for regulated release or rather for a hydrogel capable of inducing coagulation in a temperature dependent manner.

Examples of primary amine containing monomers include but are not limited to allylamine, N-3-aminopropyl methacrylamide (APM), and N-2-aminoethyl methacrylamide (AEMA). Examples of monomer units that are strong bases include those having ammonium groups, such as 3-acrylamidopropyl trimethylammonium chloride (AMPTAC). The monomer units may also be neutral monomers exhibiting no electrostatic charge in the solution. Examples of such monomers include acrylamide (Am), N-tertbutylacrylamide (NTBAAm), N-isopropylacrylamide (NIPAAm), and N,N′-dimethylacrylamide (DMAAm).

Polymerization of the monomer units can be achieved using any of various techniques known in the art, including chemical processes (e.g., using free-radical initiators and/or catalysts), photochemical processes (e.g., exposure to UV-irradiation), or electrochemical processes. Likewise, cross-linking can be achieved using any of various techniques known in the art, including the addition of a cross-linking agent to the solution. In some cases, polymerization may be effected by the addition of ammonium persulfate (APS) as the polymerization initiator and N,N,N′,N′-tetramethylethylenediamene (TEMED) as the catalyst. In some cases, the cross-linking agent is a difunctional monomer, N,N′-methylenebisacrylamide (BIS), epichlorohydrin (EPI), genipin, glutaraldehyde, or ethylene glycol diglycidyl ether (EDGE). Biodegradable cross-linkers such as ethylene glycol dimethacrylate and ethylene glycol diacrylate may also be used as the cross-linking agent. The biodegradable polymers are capable of undergoing hydrolytic cleavage in vivo. Polymerization and cross-linking may take place simultaneously or sequentially in any order. As such, the polymerization initiator, catalyst, and/or cross-linking agent may be added to the solution simultaneously or sequentially in any order.

Upon polymerization (and cross-linking, in some cases) of the monomer units, a polymer matrix is formed. The amount of cross-linker used (ratio of cross-linker monomer:total remaining functional monomers) determines the mesh size of the gel network. If a polymer hydrogel composed of a primary amine containing monomer is cross-linked appropriately the material, is capable of inducing the blood coagulation pathway, in a factor VII-tissue factor dependent manner. The ability of the polymer hydrogel to induce coagulation is dependent mainly on mechanical rigidity, i.e. cross-link density, and the primary amine functionality on the main chain polymer backbone. It should be noted that experiments were conducted using the non-cross-linked amine containing polymers, and they were unable to induce coagulation.

In certain embodiments, the polymer hydrogel is able to induce clotting in platelet deficient plasma. In other embodiments, the polymer hydrogel is able to induce clotting in Factor XII, XI, Factor IX, or Factor VIII-deficient plasma.

In a specific aspect the invention details the production of a multi-component material consisting of two different compositions of polymeric hydrogels—one for use in any internal hemostatic application, and one for use in any external hemostatic application.

In another aspect, an embodiment of the present invention provides a polymeric material comprising a cross-linked polymer matrix having a cavity, highly dense in primary amine functionality capable of inducing the blood coagulation pathway. This polymeric hydrogel may be synthesized using any of various techniques, including those described above.

The cavity may have a geometry (including its size and shape) which is capable of aiding in the activation process. Geometry of the cavity, along with density of electrostatic functional groups within the cavity, is determined, in part, by the amount of cross-linker used in the process.

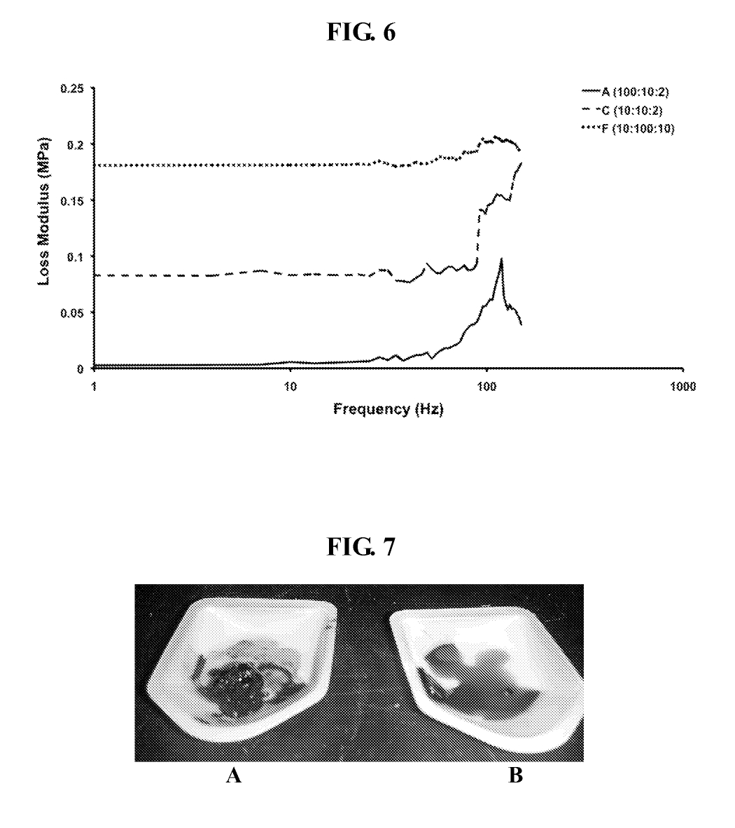

The created polymeric hydrogel, depending on the specific concentration of primary amine monomers and corresponding cross-link density may have varying degrees of inducing the blood coagulation cascade, as shown in FIG. 6 herein. In a specific embodiment, the optimum concentration for the APM, acrylamide, BIS hydrogel is between 1.5-2 M of APM and 1.5-2 M of acrylamide cross-linked at between 5:1 and 7:1 (acrylamide:BIS). In another specific embodiment (blood optimization) the optimum composition is approximately 2.73 M of APM, 0.27 M of acrylamide, and 0.056 M BIS.

The ability of the hydrogel to initiate blood coagulation in the absence of cells offers a potentially substantial advantage over other hemostatic approaches. In particular these polymers may offer treatment alternatives for patients experiencing platelet-related disorders for which there are no accepted treatment methods available. Another desirable characteristic of the materials, depicted in FIG. 1 herein, is their ability to swell in plasma. In practice this would allow the polymers to apply pressure (tamponade) at the site of action, which also aids in reducing blood loss. Furthermore, because of this characteristic swelling in plasma, the hydrogel may be designed in order to administer therapeutics in a controlled and regulated manner.

### EXAMPLES

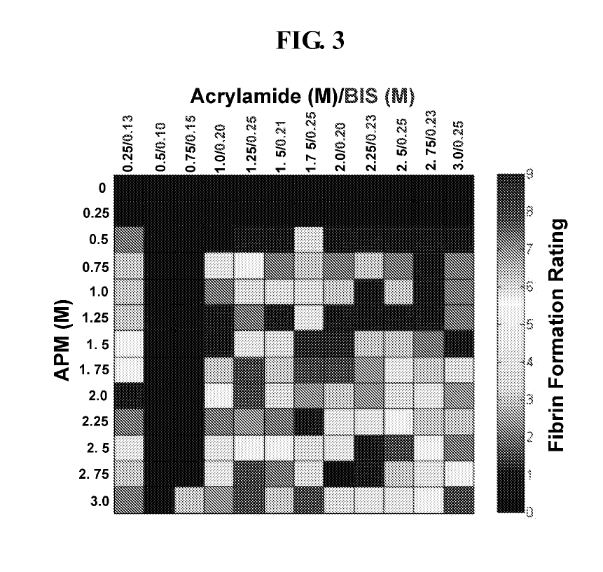

In one experiment, a randomized copolymer composed of acrylamide and APM, initiated with a 7.5% weight percent solution of TEMED (20 μL/1 mL) and a 15% weight percent solution of APS (20 μL/1 mL), and cross-linked with BIS, was produced. FIG. 3 depicts an optimization chart where 156 different polymer compositions, each cross-linked at three different ratios, were tested in citrated human plasma, and rated accordingly their ability to produce a clot. As shown the optimal concentration for inducing coagulation lies between 1.5-2 M of acrylamide and 1.5-2 M of APM, each at its highest cross-link density (maximal amount soluble). The primary amine-containing hydrogel is able to induce the blood coagulation cascade resulting in the formation of a fibrin clot. FIG. 1 depicts the ability of the material to induce a fibrin based clot in human plasma (4% sodium citrate). The hydrogel shown is composed of 1.5 M acrylamide 1.5 M APM and cross-linked with 0.3 M BIS (acrylamide:BIS ratio=5:1).

FIG. 2 shows micrographs of the coagulate complex (fibrin-hydrogel complex) after hematoxlyin and eosin (H&E) staining, immunohistochemical (IHC) staining, along with an image of the complex obtained using an environmental scanning electron microscope.

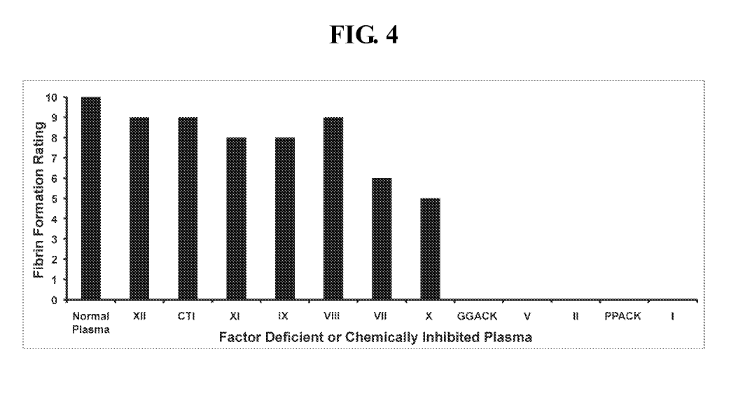

FIG. 4 shows the ability of the optimized hydrogel (1.5 M acrylamide, 1.5 M APM< and 0.3 M BIS) to induce fibrin formation in a variety of factor deficient and factor inhibited plasmas.

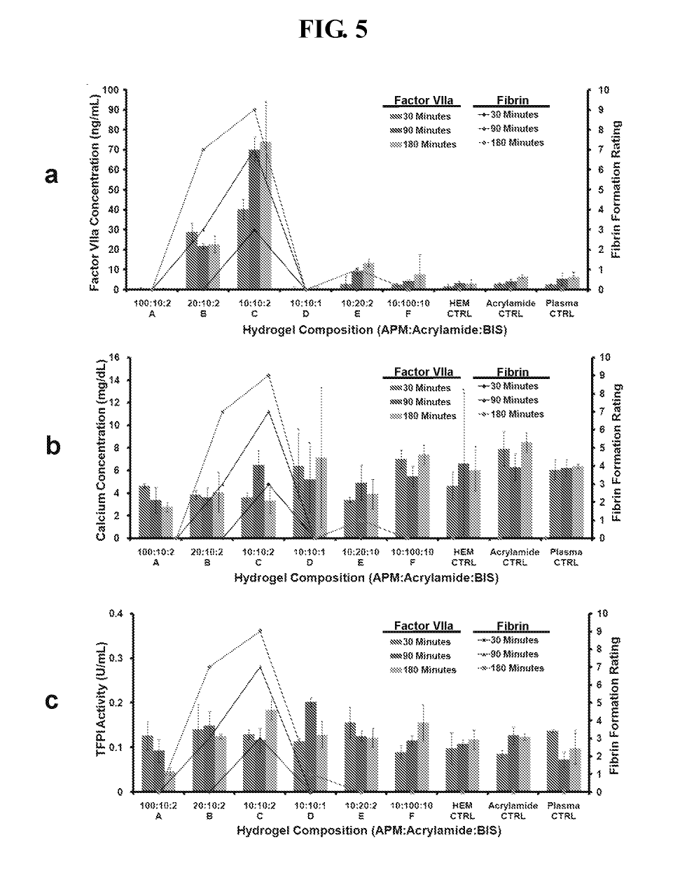

FIG. 5 shows the ability of the optimized hydrogel to induce the activation of FVII (5C).

FIG. 6 shows that the optimized hydrogel is not in fact a homogenous network but rather is made up of several mechanically distinct regions.

Experimentation in human and sheep blood produced an optimum composition consisting of 0.27 M of acrylamide and 2.73 M of APM and 0.056 M (acrylamide:BIS ratio=5:1). [Composition 2]

The effectiveness of the primary amine-containing hydrogel in hemostatic clot formation was assessed. Blood was drawn from a live adult sheep and added immediately to a vial containing a small amount of the primary amine-containing hydrogel (FIG. 7A) and an empty vial (FIG. 7B), used as a control. The material was able to induce the formation of a robust clot within seconds of blood contact, compared to the control which took approximately 10 minutes. Furthermore, the mechanical integrity of the clot produced was dramatically superior to that of the control.

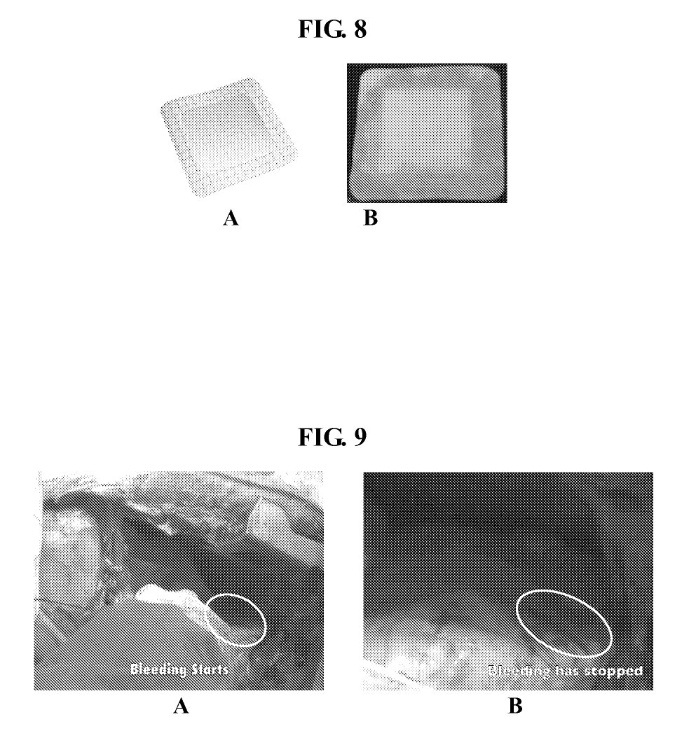

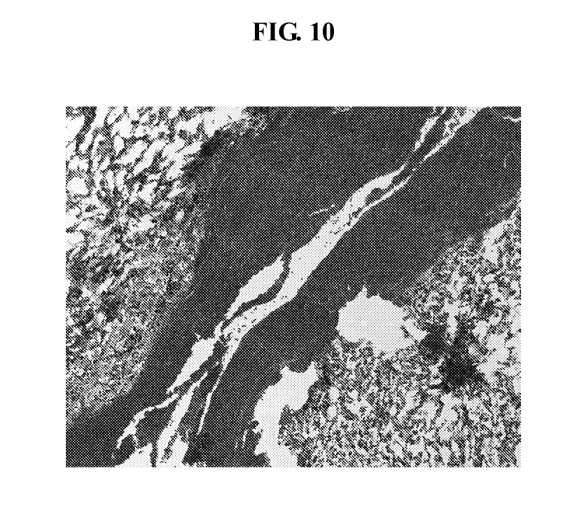

The primary amine-containing hydrogel was also effective in inducing hemostatic clot formation in vivo. A primary amine-containing hydrogel, consisting of 0.27 M acrylamide, 2.73 M N-3-aminopropyl methacrylamide (APM), and 0.054 M N-N′-methylene bisacrylamide (BIS), placed on a 4×4 inch gauze bandage (a prototype of the presently claimed invention, shown in FIG. 8), was administered to inhibit bleeding from an incision introduced into a live sheep lung. The hydrogel was able to successfully stop bleeding from the induced lung incision in approximately 2 minutes. FIG. 9 shows an image of the site immediately after the surgeon made the incision (FIG. 9A) along with an image of the incision site after the hydrogel based prototype was applied for 2 minutes (FIG. 9B). Initial post operative analysis showed that the material was able to induce fibrin formation at the incision causing a natural suturing process, and thus sealing the tissue preventing blood loss. Hematoxylin and eosin stained sections of the incision site confirmed that the material was able to induce the rapid formation of a natural, fibrous-based hemostatic suture, as shown in FIG. 10.

### INDUSTRIAL APPLICABILITY

The polymer hydrogel created of the present invention may have various uses. Such uses include a bandage for trauma related injuries or a surgical gauze for use in the operating rooms. In terms of the bandage the hydrogel would be incorporated into a filtered bandage, similar to that of a Band-Aid®, which would then be applied to the wound in order to prevent blood loss and deliver necessary therapeutics. In terms of the surgical gauze the hydrogel would be incorporated into a filtered gauze-like material for use by surgeons to control blood loss during surgery.

Notably, the bandage application of the hydrogel is novel in the sense that there is no other synthetic polymer, hydrogel material capable of inducing blood clotting while simultaneously delivering therapeutics. Furthermore, the hydrogel functions in Factor VIII and Factor IX deficient plasma, a functionality which should revolutionize wound care for people suffering from hemophilia.

1. A cross-linked polymer, comprising cross-links between at least two monomer backbones, wherein at least one monomer comprises a primary, secondary, tertiary or quaternary amine with a pKa of at least 7.4, and capable of displaying a positive electrostatic charge at the pH of blood or plasma (7.4), and wherein the polymer is capable of activating the blood coagulation cascade by inducing fibrin formation.

2. The polymer of claim 1, wherein said at least one monomer is selected from the group consisting of N-3-aminopropyl methacrylamide, allylamine, and a primary-amine containing monomer capable of displaying a positive electrostatic charge at the pH of blood or plasma (7.4).

3. The polymer of claim 1, wherein at least one of the monomer backbones is acrylamide, or a derivative thereof.

4. A hydrogel comprising the polymer of claim 1, wherein the monomer backbones are cross-linked with cross-linkers selected from the group consisting of N-N′-methylene bisacrylamide, N-N′-bisacrylylcystamine, bisacrylyl piperazine, ethylene glycol diglycidyl ether, epichlorohydrin, N-N′-diallyltartardiamide, ethylene glycol dimethacrylate and ethylene glycol diacrylate.

5. The hydrogel of claim 4, wherein the primary amine is allylamine and the cross-linker is either ethylene glycol diglycidyl ether or epichlorohydrin.

6. The hydrogel of claim 4, therein the monomer backbones are cross-linked with cross-linkers selected from the group consisting of ethylene glycol dimethacrylate and ethylene glycol diacrylate.

7. The polymer of claim 1, wherein the primary amine containing monomer is N-3-aminopropyl methacrylamide (APM), the co-monomer is acrylamide, and the cross-linker is N-N′-methylene bisacrylamide (BIS).

8. The hydrogel of claim 4, wherein said hydrogel consists of 1.5 M acrylamide, 1.5 M N-3-aminopropyl methacrylamide (APM), and 0.3 M N-N′-methylene bisacrylamide (BIS).

9. The polymer of claim 7, wherein said hydrogel consists of 0.27 M acrylamide, 2.73 M N-3-aminopropyl methacrylamide (APM), and 0.054 M N-N′-methylene bisacrylamide (BIS).

10. The hydrogel of claim 4, wherein said hydrogel consists of 1.5 M acrylamide, 1.5 M N-3-aminopropyl methacrylamide (APM), and 0.3 M N-N′-methylene bisacrylamide (BIS).

11. The hydrogel of claim 4, further comprising one or more pharmaceutical active ingredients selected from the group consisting of Novocain, Lidocain, erythromycin, bacitracin, adrenaline, topricin, acetaminophen, ibuprofen, and a hemostatic agent.

12. A method of forming the hydrogel of claim 4, comprising:
adding the monomer backbone comprising a primary amine to the aqueous solution, wherein the first monomer unit exhibits an electrostatic positive charge in the aqueous solution at pH of 7.4 (pH of blood or plasma);
adding a second monomer backbone to the aqueous solution, wherein the second monomer backbone is different from the first monomer unit;
forming the cross-linked polymer using a cross-linking agent, wherein the step of forming polymer comprises polymerizing the monomer backbone.

13. The method of claim 12, wherein the second monomer backbone is neutral or exhibits an electrostatic charge opposite to that of the first monomer backbone.

14. The method of claim 12, wherein the first monomer backbone is added before the second monomer backbone is added.

15. The method of claim 12, wherein the second monomer backbone is added before the first monomer backbone.

16. The method of claim 12, wherein the first and second monomer backbone are added simultaneously to the aqueous solution.

17. A kit for inducing hemostatic clot formation, comprising the polymeric hydrogel of claim 4.

18. A kit for inducing hemostatic clot formation, comprising the polymeric hydrogel of claim 5.

19. A kit for inducing hemostatic clot formation, comprising the polymeric hydrogel of claim 6.

20. A kit for inducing hemostatic clot formation, comprising the polymeric hydrogel of claim 8.

21. A kit for inducing hemostatic clot formation, comprising the polymeric hydrogel of claim 10.

22. A method of treating trauma-induced hemorrhage, comprising administering the hydrogel of claim 4 to a subject in need thereof.

23. A method of treating internal hemorrhaging, comprising administering the hydrogel of claim 4 to a subject in need thereof.

24. A method of treating hemorrhaging during surgical operations, comprising administering the hydrogel of claim 4 to a subject in need thereof.

25. The method of claim 22, wherein said hydrogel is administered on a material selected from the group consisting of a bandage, gauze, tape and adhesive wound dressing.
